(19) United States
(12) Patent Application Publication
(10) Pub. No.: US 2008/0227222 A1
(43) Pub. Date: Sep. 18, 2008

(54) Method for the detection of target molecules by fluorescence polarization using peptide mimics

(76) Inventors: Malford E. Cullum, Grayslake, IL (US); Karen H. O'Connor, Evanston, IL (US)

Correspondence Address: NAVAL MEDICAL RESEARCH CENTER;ATTN: (CODE 00L), 503 ROBERT GRANT AVENUE, SILVER SPRING, MD, 20910-7500, US

(21) Appl. No.: 11/726,203

(22) Filed: Mar. 13, 2007

(51) Int. Cl. G01N 33/533; G01N 33/53; G01N 33/566; G01N 33/531

(52) U.S. Cl. 436/546; 435/5; 435/7.2

(57) ABSTRACT

The inventive subject matter relates to a method of measuring non-protein hydrophobic and protein target antigens by competitive fluorescence polarization using peptide mimics as competitors. The peptide mimics used in the inventive method contain conformational epitopes not represented in the linear sequence. The method can be used in the detection of a wide range of agents including steroids, hormones, nucleic acids, proteins and infectious organisms. A method for the detection of cortisol and gamma-interferon is also contemplated in the method. Samples suitable for analysis by the inventive method include environmental samples and bodily fluids including serum and oral fluids including saliva.

### SEQUENCE LISTING

I hereby state that the information recorded in computer readable form is identical to the written sequence listing.

### BACKGROUND OF INVENTION

1. Field of the Invention

The inventive subject matter relates to a competitive fluorescence method for estimating the concentration of hydrophobic molecules and proteins in a wide range of sample matrixes by fluorescence polarization using a peptide mimic as competitor.

2. Description of the Related Art

Fluorescent polarization (FP) technology permits rapid, real-time, sensitive evaluation of fluid phase antigens with high specificity (Kowski and Wu, 2000). FP is predicated on the principle that fluorescent molecules emit polarized fluorescence when they absorb polarized light at a specific wavelength.

Inherent in molecules in solution is their tendency to rotate. When polarized light strikes the molecules in solution, the emitted light does not remain polarized because the molecule is rotating rapidly in solution. Therefore, in FP based assays, polarized incident visible or ultraviolet light that illuminates a fluorochrome causes subsequent polarized fluorescence with emission at a longer wavelength. However, molecules in solution are capable of free rotation. Polarized light striking a fluorescent molecule loses polarization due to rotation of the molecule with the rate of rotation dependent on the size of the molecule.

Therefore, solutions containing slower turning, large molecule-fluorochrome complexes tend to stay polarized longer compared to smaller labeled molecules. Therefore, an antigen/antibody complex will have an inherently slower rate of rotation than antigen alone causing more of the polarized fluorescence to be emitted in the same plane as the incident light. In order to accommodate molecules of different sizes (up to 10⁷ kDa molecular weight), different fluorochromes can be selected (Terpetschnig, et al, 1995).

Combining a fluorochrome-labeled antigen or peptide with antibody results in an increase in FP, as measured in arbitrary millipolarization (mP) units. The smaller the fluorescent antigen, the greater the increase in mP units that is measured upon binding to its corresponding antibody, since mP depends upon the partial specific volume (approximate molecular weight in solution) of the labeled substance. The dependence is non-linear but is describable in a Perrin equation.

FP antigen-antibody direct binding assays require only the mixing of fluorescent reagent (antigen) with the sample (containing antibody) in a liquid buffer. In an FP assay, minimally two FP readings are necessary, a base-line reading and a reading after a specified time. In a direct binding assay, the FP value increases as binding of antigen and antibody occurs. The difference in FP between a fluorescent antigen of 10 kDa initially and the fluorescent complex consisting of it and IgG, for example, results in a measurable association, using less than saturating antibody concentrations (Tencza, et al, 2000).

Because FP is conducted in a fluid environment and because polarization is a general property of fluorescent molecules, FP assays have the potential to be less susceptible to non-specific interactions occurring at the cell surface and to interferences present in non-homogeneous sample fluids. Furthermore, salivary and oral fluid assays, as opposed to the use of serum, have been increasingly recognized as a better, non-invasive alternative to serum-based diagnostics in detecting certain hormone, drug, antibody and antigen detection (Rossomando, et al, 2001; Tabak, 2001).

Other advantages of FP technology are that FP assays can accommodate relatively cloudy solutions, such as oral fluids or serum, without the need for time-intensive or expensive purification or clarification schemes. Furthermore, FP assays can be designed to accommodate significant variation in pH in fluid samples, such as in some media or in saliva or urine, by utilizing different pH-independent fluorochromes (Schade and Jolley, U.S. Pat. No. 5,804,395).

Despite clear advantages to the use of FP assay technology, difficulty exists in designing competitive FP assays utilizing competitor molecules over 10 KDa. A serious limitation of FP assays is the quantitatable change in size between unbound probe and bound probe when the probe is >10 k Da in size. This limitation is imposed by the lack of any substantially useful dyes with very large emission lifetimes (tau) greater than 100 nanoseconds with a quantum efficiency equivalent to that of fluorescene. Although many attempts have been made to discover or invent substitutes for fluorescence or similar dyes (Terpetschnig, et al, 1995).

Additionally, sensitive detection of highly hydrophobic moieties, such as steroidal compounds, is often difficult due to non-specific interaction of the target molecule with unrelated proteins in the sample. This anomaly is increasingly important due to the greater recognition that oral fluids, in addition to serum, are acceptable, an at times preferred, alternatives for use in diagnostic tests for certain hormones, drugs, antibodies, antigens and stress markers (Kraus and Konno, 1965; Hirschman and Kresge, 2001; Hofman, 2001; Tenovuo, 1989; Gallo, et al, 1997; Thieme, et al, 1994; Kirschbaum and Hellhammer, 1994).

Epitope mapping has been an important approach in elucidating critical regions of complex proteins for efficacious vaccine construction and assay design (Rowley, et al, 2004). Furthermore, the use of peptides derived from the native amino acid sequences of proteins has been described. For example Buchli, et al (2005) describes the use of FP for the development of competitive FP assays for HLA.

Peptides with sequences that can mimic an epitope without homology to the original antigen sequence have been selected (Geysen, et al, 1986; O'Connor, et al, 2005). However, despite the critical need for small competitor molecules in order to fully appreciate the potential of FP technology, the use of mimics, containing conformational epitopes of the target moiety, has not been integrated into FP-based assays.

### SUMMARY OF THE INVENTION

An object of the invention is an assay method for the detection and quantitation of target antigens, including proteins and hydrophobic molecules in a variety of samples including food and environmental samples as well as body fluids such as serum, oral fluids and saliva by competitive fluorescent polarization (FP) utilizing a peptide mimic as competitor. The peptide mimics incorporate important target epitopes, including conformational epitopes. The inventive method permits detection and quantitation of a large array of target molecules, including those over 10 kDa.

Another object is a method for the detection of cortisol by competitive FP using a peptide mimic as competitor. The inventive method includes peptide mimic sequence motifs and specific peptide mimic sequences that contain important cortisol epitopes.

A still further object is a method for the detection of gamma-interferon by competitive FP using a peptide mimic as competitor. The inventive method includes a peptide mimic sequence and specific peptide mimic sequences that contain important epitopes, including conformational epitopes.

### BRIEF DESCRIPTION OF THE DRAWINGS

FIG. 1. Illustration of the general scheme of the inventive competitive fluorescence polarization assay.

FIG. 2. Biopanning results using anti-cortisol antibody (ACA) in ELISA.

FIG. 3. Binding of cortisol phage ACA using increasing amounts of cortisol and constant concentration of phage containing mimic.

FIG. 4. Illustration of graded competition for binding to anti-gamma-interferon antibody with increasing amounts of gamma-interferon and constant concentration of phage containing peptide mimic (IFN-28).

FIG. 5. Gradient inhibition of IFN-41 and IFN-19 using anti-gamma-interferon antibody.

### DETAILED DESCRIPTION OF PREFERRED EMBODIMENTS

Competitive fluorescence polarization technology has many advantages over other methods in detection and diagnostic assays including reduced interference from sample turbidity. However, despite the inherent advantages of FP technology, a serious disadvantage is the need for relatively small competing moieties. All available FP assays are used for small molecules, since small molecule competitors are required. Although peptide fragments of proteins can be used as competitors, antibody variable regions often don't recognize linear peptide sequences, but rather they tertiary protein structures. This is especially true of higher affinity protein-antibody interactions.

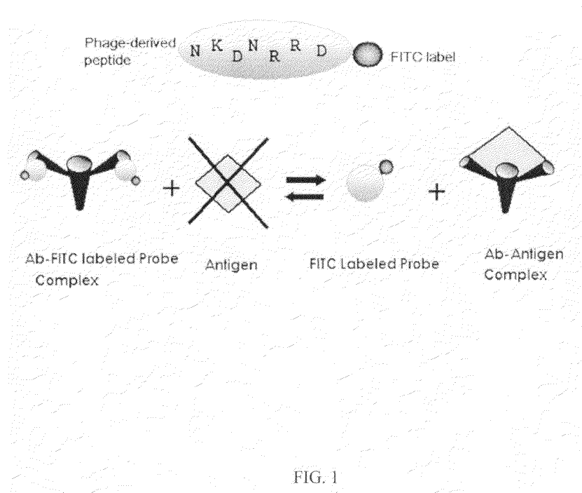

To address this issue, a competitive fluorescence polarization immunoassay (CFPIA) method utilizing peptide sequences that could potentially mimic relevant target epitopes. Therefore, an aspect of the current invention is the use of peptide mimics in competitive FP assay for the detection of target antigens greater than 10 k Da. The general scheme of the inventive CFPIA is illustrated in FIG. 1. As shown in FIG. 1, in place of using the target molecule as a competitor, a smaller peptide mimic, encompassing important antibody epitopes, is substituted. The peptides would be selected based on their ability to directly compete for the binding of specific antibody with target moiety, such as whole protein or large protein fragments containing the specific binding regions. Because the peptide mimics are small, the inventive CFPIA is capable of being utilized for a wide range of molecules, including steroids, hormones, nucleic acids and a wide MW range of proteins.

Selection of peptide mimics is predicated on their ability to effectively compete for binding to specific antibody with the target molecular moiety. However, an important aspect of the current invention is the use of mimics in FP assays that are not necessarily reminiscent of the linear epitopes, but rather also include those peptides sequences not found in represented in the native protein but nevertheless contain important epitopic fingerprints.

The fingerprint represents relevant contact residues that reproduce or mimic important antibody-recognized epitopes. In fact, the amino acids contained in a particular mimic may include those that may be spatially distant in the native protein or target molecule but that when all the amino acids in the mimic are represented together create a fingerprint that is reminiscent of conformational epitopes of the target protein or other molecule. Conformational epitopic sites are often of greatest binding affinity, compared to linear epitopes. Use of the inventive method, therefore, permits the evaluation of target antigens larger than 10 kDa by FP assay technology.

An additional aspect of the inventive method addresses the issue that some targets, especially steroidal targets like cortisol, often possess inherent hydrophobicity leading to non-specific adherence of the steroid molecule to protein species present in the sample. Table 1 illustrates the effect of inherent hydrophobicity of a compound, such as cortisol, on assay sensitivity. As seen in Table 1, non-specific adherence of cortisol greatly reduces assay sensitivity, yielding a concentration determination of salivary cortisol of 10 ng/ml. The clinically relevant concentration of salivary cortisol is 0-8 ng/ml, which is well within the range of ELISA and FP-based assays. Only by the addition of solubilizing detergent is cortisol detectable.

TABLE 1

| | | | Actual Cortisol |
|---|---|---|---|
| | | Concentration | Concentration |
| | Polarization | (based on FP) | (based on ELISA) |
| Assay Condition | Value | ng/ml | ng/ml |
| Cortisol-fluoroscein | 320 | 0 | 10 |
| label | | | |
| Cortisol-fluoroscein | 280 | 10 | 10 |
| label plus detergent | | | |

Therefore, an aspect of the current invention is the use of peptide mimics in competitive FP assays in order to abrogate the reduction in assay sensitivity due to hydrophobic properties surrounding certain target molecules such as steroidal compounds. For these assays, instead of utilizing labeled target molecule as a competitor, amino acid sequences are selected which are operationally capable of specifically competing for binding with specific antibody.

In addition to the improved operational advantages of the inventive method, the method also provides for increased safety of assay operation. This is particularly acute when in situations were the target antigens are infectious organisms, such as Mycobacterium tuberculosis, Bacillus anthracis, influenza strains and dengue or toxins such as botulinum toxin, sarin, and afalotoxin. Furthermore, kits including peptide mimic competitive reagent would be more stable and possess a greater shelf life than if intact target agent or proteins were required as competitor.

The basic procedure for measuring target molecules by FP is essentially as follows, using fluorochrome-labeled peptide mimic as the competitive reagent and either polyclonal or monoclonal antibody specific to target molecule:
a. intermixing a saliva, serum or other fluid sample and a fluorochrome-labeled-peptide mimic (i.e. competitor) with a target molecule-specific monoclonal or polyclonal antibody or other ligand. Typically, 5-20 μl of sample are added to 0.2 to 1 ml of buffer;
b. incubating the competitive peptide mimic and specific antibody. The incubation time varies, depending on target and suspected concentration of target antigen in the sample. However, typically, incuabation times range from 15 seconds to 5 minutes;
c. detecting the binding interaction of the target molecule and specific antibody by horizontal and vertical measurement, typically in mP;
d. quantitating the concentration of the target molecule as a result of the interaction of peptide mimic and specific antibody.

The general scheme is applicable to the FP detection of hydrophobic moieties such as cortisol, hormones, toxins and drugs. Additionally, the scheme is applicable to the detection and quantitation of other large protein antigens or infectious agents.

Different fluorochromes can be utilized in order to optimize results, including the incorporation of pH-independent fluorochromes. The fluorochromes that are included as an aspect of the invention include: 7-AAD, Acridine Orange, Alexa 488, Alexa 532, Alexa 546, Alexa 568, Alexa 594, Aminonapthalene, Benzoxadiazole, BODIPY 493/504, BODIPY 505/515, BODIPY 576/589, BODIPY FL, BODIPY TMR, BODIPY TR, Carboxytetramethylrhodamine, Cascade Blue, a Coumarin, Cy2, CY3, CY5, CY9, Dansyl Chloride, DAPI, Eosin, Erythrosin, Ethidium Homodimer II, Ethidium Bromide, Fluorescamine, Fluorescein, FTC, GFP (yellow shifted mutants T203Y, T203F, S65G/S72A), Hoechst 33242, Hoechst 33258, IAEDANS, an Indopyras Dye, la Jolla Blue 3, a Lanthamide Chelate, a Lanthamide Cryptate, Lissamine Rhodamine, Lucifer Yellow, Maleimide, MANT, MQAE, NBD, Oregon Green 488, Oregon Green 514, Oregon Green 500, Phycoerythrin, a Porphyrin, Propidium Iodide, Pyrene, Pyrene Butyrate, Pyrene Maleimide, Pyridyloxazole, Rhodamine 123, Rhodamine 6G, Rhodamine Green, SPQ, Texas Red, TMRM, TOTO-1, TRITC, YOYO-1, vitamin B12, flavin-adenine dinucleotide, and nicotinamide-adenine dinucleotide.

To better illustrate the invention, the following examples are disclosed, including the development of a competitive reagent for cortisol and gamma-interferon. However, it is contemplated that the invention would encompass assays for other compounds including hormones, toxins and drugs and for the FP detection and quantitation of other proteins, including large (>10 KDa) molecules.

The inventive method is also applicable to the detection of infectious organisms such as bacteria and viruses. This aspect is especially important since a serious limitation of FP assays is the quantitatable change in size between unbound probe and bound probe when the probe is >10 k Da in size due to a lack of dyes with large emission lifetimes (tau) greater than 100 nanoseconds and with a quantum efficiency equivalent to that of fluorescene. Therefore, the ability to map a small epitope off the surface of a large protein, virus or bacteria is important in order to broaden the scope of use of FP assay technology.

### EXAMPLE 1

### Method for the Development of Peptide Mimics

Many important target antigens contain complex epitopes; include conformational epitopes with binding sites that are linearly located significant distances apart. Furthermore, many important cytokines are expressed as multimers, e.g., dimers, making identification of epitopes by sequence walking impossible.

An inventive aspect of the current invention is a method for the identification of peptide mimics by phage display technology. Phage-displayed peptide libraries, each expressing peptides at the N-terminus of the five copies of the pIII coat protein of the filamentous coliphage M13, were purchased from New England Biolabs (Beverly, Mass.). These included a heptapeptide library, heptapeptide (cysteine constrained) library and a dodecapeptide library. The libraries contained 2×10¹³ pfu/ml and were used at a concentration of 8×10¹⁰ pfu/ml. Phage was propagated in Escherichia coli strain ER2537.

The libraries were screened by biopanning using anti-target antibody and negative selection using only the biopanning materials without the target antibody in order to remove phage that bound nonspecifically. The pIII libraries were screened using 3 μl of each library and approximately 100 μg of antibody, depending on the antibody, in phosphate buffered saline (PBS), pH 7.3, supplemented with 1 mg/ml BSA. The mixture was left overnight at 4° C. and phage-antibody complexes were isolated using paramagnetic beads coated with anti-mouse IgG. The beads were washed 10 times in PBS-BSA, and phage were eluted from the antibodies with 100 μl of 0.1 M HCl adjusted to pH 2.2 with glycine and neutralized immediately with 40 μl of 1 M Tris-HCl pH 9.9. ER2537 bacterial cells in log phase were infected with the eluted phage and the culture was grown for 4.5 h. The bacterial cells were pelleted twice by 15 min centrifugations at 4,000×g. Phage was precipitated from the supernatant using 25% polyethylene glycol (PEG) 6000 and 2.5 M NaCl on ice for 1 hour and pelleted by centrifugation for 15 min at 10,000×g. The pelleted phage was resuspended in 10 mM Tris, 1 mM EDTA, pH 8. Precipitation was repeated and the final phage pellet was resuspended in PBS and used for further rounds of biopanning. Three rounds of positive selection were performed, and negative selection was performed after the first and second rounds of positive selection, each followed by amplification. Phages were used to infect the host bacterial strain and single phage clones were isolated.

Identification of reactive phagotopes was conducted by either direct, where the phagotopes are coated directly to the plate, or capture ELISA, were the phagotopes are tethered to the plate via the selection antibody. The two ELISA methods were employed because direct ELISA identifies highly reactive phagotopes that are likely to contain a strong motif, whereas the capture ELISA is more sensitive, therefore also selecting for phagotopes involving only weak interactions. For the direct ELISA, phage preparations were diluted in sterile PBS and duplicate samples of 100 μl/well were added to a 96-well microtitre plate. Plates were left overnight at 4° C., blocked for 2 hours at room temperature with 1% skimmed milk powder and 0.05% Tween 20 in PBS ph 7.4 and washed three times in Tris-buffered saline, pH 7.4, containing 0.05% polyoxyethylene (20) sorbitan monolaurate (TWEEN 20™). 100 μl of specific antibody diluted to 1 μg/ml in 1% PBS-polyoxyethylene (20) sorbitan monolaurate were added and left overnight. The plates were washed and exposed to horseradish peroxidase (HRP)-conjugated anti-mouse Ig diluted in PBS without polyoxyethylene (20) sorbitan monolaurate for 2 hours, washed six times and developed with 0.5 mg/ml 2,2-azino-di-(3-ethyl-benzthiazoline 6 sulfonate) (ABTS) in 0.03 citric acid, 0.04 M Na₂HPO₄, pH4 and 0.003% H₂O₂. Absorbances were read at 415 nm after 30 minutes and 60 minutes. Phage with stop codons or no inserts were included in each assay and were used as negative controls.

For capture ELISA, phage was captured by specific antibody immobilized onto microtitre plates. Bound phage was detected using polyclonal sheep antibodies to M13 phage and HRP-conjugated anti-sheep/goat Ig. Wells of microtitre plates were coated with 100 μl/well of the selecting mAb diluted to 1 μg/ml in PBS pH 7.4, left at 4° C. overnight, blocked and washed. Duplicate samples of 5 μl of phage diluted in sterile PBSA were added and left overnight at 4° C. The plates were blocked and washed before adding 100 μl of antibody to M13 phage diluted in PBS-polyoxyethylene (20) sorbitan monolaurate. Phage bound by the antibody were detected by exposure to HRP-conjugated anti-sheep/goat antibody diluted in PBS for 2 hours followed by development with 0.5 mg/ml ABTS in 0.03 M citric acid, 0.04 M Na₂HPO₄, pH4 and 0.003% H₂O₂.

Subsequent to panning, phagotopes of interest were selected for sequencing. Single-stranded DNA was prepared by phenol extraction and sequenced using the Sequenase™ version 2.0 (T7 DNA sequencng kit (Amersham, Cleveland, Ohio). The M13 (−28 gIII) sequencing primers supplied were used, and [α-³⁵ S]-dATP Redivue™ (Amersham) was used as label for phagotypes selected from the libraries.

Sequencing was conducted using dGTP and ambiguous sequences were resequenced using the nucleotide analogue dITP to minimize secondary structure formation. Samples were separated by electrophoresis using a 4.5% acrylamide denaturing gel.

### EXAMPLE 2

### Development of Competitive Reagent Against Cortisol

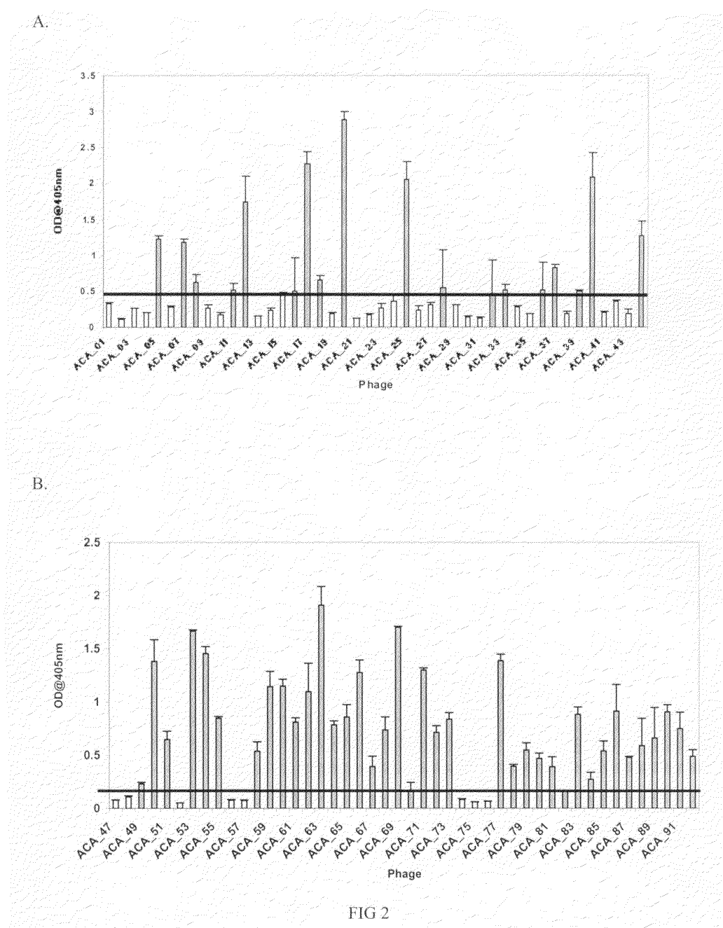

Identification of cortisol mimics was essentially conducted as described in Example 1. For cortisol, the libraries were screened by biopanning using anti-cortisol antibody and negative selection using only the biopanning materials without the target antibody in order to remove phage bound nonspecifically, as described in Example 1. FIG. 2 illustrates the biopanning results using anti-cortisol antibody (ACA) in ELISA. In FIG. 2, those phages yielding an OD (405 nm) below the threshold denoted by the solid horizontal line were considered negative.

Plates were coated with phagotopes and blocked as for the direct ELISA. For these studies, phagotopes expressing peptides that exhibited comparatively high anti-cortisol antibody binding, ACA 20 (1) and ACA 20 (2), were selected for initial studies. As a control, ACA 52 was selected, which was selected using a different antibody and therefore does not bind to ACA. Increasing amounts of cortisol was then added to 100 μl of specific antibody in 1% PBS-polyoxyethylene (20) sorbitan monolaurate. 100 μl replicates (4-6) of antibody/cortisol inhibitor was then left overnight.

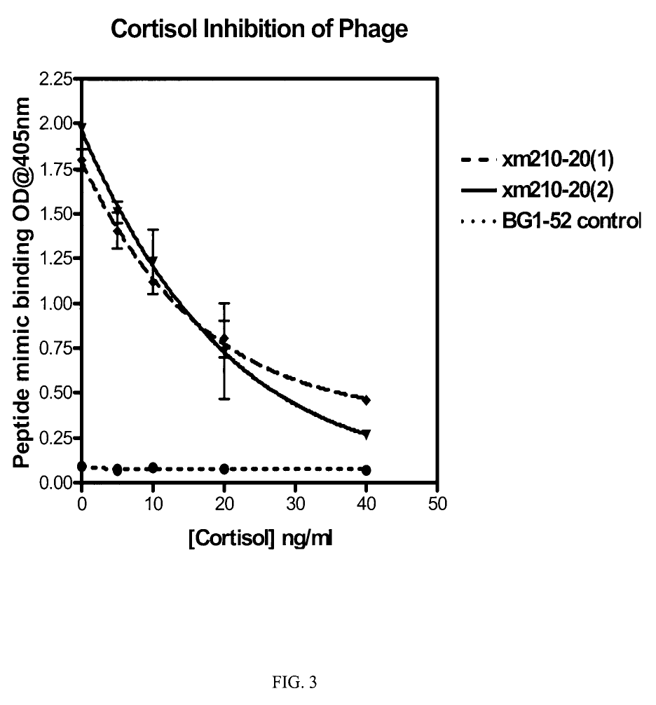

The plates were washed and developed as for the direct ELISA. The results of these studies are illustrated in FIG. 3. In FIG. 3, binding of ACA 20 (1) and ACA20 (2) exhibited overlapping, dose-response competitive binding curves. No competition was exhibited in the face of the control ACA 52, expressing an irrelevant peptide sequence.

Table 2 illustrates the sequence results of identified cortisol peptide mimics. Those sequences with “c” as a suffix include cyteine in order to circularize the peptide. The selected cortisol peptide mimics are generally represented by the motifs P+T+Y (or F) (some mimics also contained either W+N or L+R); W(F)+N(Q); or L(I)+R(H/K).

TABLE 2

| Mimic | Peptide | | |
|---|---|---|---|
| clone | Sequence | SEQ ID No. | Motif |
| ACA-71c | AC-MLSSQTLH-C | SEQ ID No. 25 | L(I) + R(H/K) |
| ACA-5 | LFQTTVRTHALF | SEQ ID No. 26 | L(I) + R(H/K) |
| ACA-61 | GYQPLRH | SEQ ID No. 27 | W(F) + N(Q) or |
| | | | L(I) + R(H/K) |
| ACA-68 | AHNLNYKPLRHP | SEQ ID No. 28 | L(JI)+ R(H/K) |
| ACA-72 | KLMVPLH | SEQ ID No. 29 | L(I) + (H/K) |
| ACA-40 | FAHPLYP | SEQ ID No. 30 | L(I) + R(H/K) |
| ACA-66 | FVPSPHRPLYQP | SEQ ID No. 31 | L(I) + R(H/K) |
| ACA-63c | AC-TQFNLHY-C | SEQ ID No. 32 | W(F) + N(Q) |
| ACA-69 | WPFTYHMPTHYG | SEQ ID No. 33 | P + T + F(Y) |
| ACA-50 | LPYNVHWAKGRP | SEQ ID No. 34 | L(I) + R(H/K) |
| ACA-64 | GPSTFFSSGLLR | SEQ ID No. 35 | P + T + F(Y) |
| ACA-73 | MGPSNMY | SEQ ID No. 36 | |
| ACA-37c | AC-PATSIRM-C | SEQ ID No. 37 | L(I) + R(H/K) |
| ACA-55 | SQNIFRW | SEQ ID No. 38 | W(F) + N(Q) |
| ACA-87 | QNNHMSSRQWFA | SEQ ID No. 39 | (W(F) + N(Q) |
| ACA-12c | AC-SQFNSLF-C | SEQ ID No. 40 | W(F) + N(Q) |
| ACA-7c | AC-SQYNRLV-C | SEQ ID No. 41 | L(I) + R(H/K) |
| ACA-53c | AC-TLPWSNS-C | SEQ ID No. 42 | W(F) + N(Q) |
| ACA-85 | TNLYQDKPSNRS | SEQ ID No. 43 | L(I) + R(H/K) |
| ACA-62 | LWPRSDSHIPAF | SEQ ID No. 44 | L(I) + R(H/K) |
| ACA-90 | KNWNTPTYYSSM | SEQ ID No. 45 | W(F) + N(Q) |
| ACA-65 | SKNWHTPTYSTL | SEQ ID No. 46 | L(I) + R(H/K) or |
| | | | W(F) + N(Q) |
| ACA-67 | LSKNWQT | SEQ ID No. 47 | W(F) + N(Q) |
| ACA-17c | AC-SQWSNVL-C | SEQ ID No. 48 | W(F) + N(Q) |
| ACA-25c | AC-TQWSQLY-C | SEQ ID No. 49 | W(F) + N(Q) |
| ACA-77 | WYSPEYYVLPSL | SEQ ID No. 50 | W(F) + N(Q) |
| ACA-18c | AC-PPPWHTM-C | SEQ ID No. 51 | |
| ACA-86c | AC-VNPNLKV-C | SEQ ID No. 52 | |
| ACA-83c | AC-INPNDKK-C | SEQ ID No. 53 | |
| ACA-58 | TASLTKTSVNPK | SEQ ID No. 54 | P + T + F(Y) |
| ACA-20 | YFTPVFINPTQV | SEQ ID No. 55 | P + T + F(Y) |
| ACA-60 | LPKSSLQALQLR | SEQ ID No. 56 | L(I) + R(H/K) |
| ACA-59 | LQVNLEQDKLRP | SEQ ID No. 57 | L(I) + R(H/K) |
| ACA-44 | LDLRQNSSPPLR | SEQ ID No. 58 | L(I) + R(H/K) |
| ACA-54 | LHTQIIAFTEGN | SEQ ID No. 59 | L(I) + R(H/K) |
| ACA-91 | HVNSSISGQLL | SEQ ID No. 60 | L(I) + R(H/K) |

As an example of the use of mimics in competitive FP assays, a method for the detection of the steroid cortisol using peptide mimics is illustrative. The assay can be conducted using either serum or oral or salivary fluid samples. Unlike total serum cortisol, which is composed of a combination of “non-active” carrier-bound and free cortisol, salivary cortisol comprises “biologically active” free cortisol. Because salivary cortisol is equilibrated into the salivary extravascular pool, its concentration is independent of saliva flow and is proportional to circulating levels making it an ideal candidate for non-invasive sampling (Kirschbaum and Hellhammer, 1994).

The current method permits measurement of cortisol concentrations in a fraction of the time required for ELISA. The general scheme is illustrated in FIG. 1. The labeled cortisol peptide mimic will produce polarized fluorescence if correctly aligned when excited by polarized light of the appropriate wavelength. However, the detector will capture a small portion of the fluorescence because cortisol is so small. Small molecules change their orientation rapidly and become depolarized. However, encumbering the cortisol peptide mimic, for example by attaching a large antibody molecule, will increase the amount of fluorescence detected. The subsequent addition of unlabeled cortisol, such as from samples of serum, plasma, milk, urine or other sources, will decrease the amount of polarized fluorescence detected proportionate to the amount of unlabeled cortisol added.

The cortisol peptide mimics identified in Table 2 can be utilized in the inventive competitive FP assay for detecting and quantitating cortisol or corticosterone. The general assay method is as follows:
a. Incubate collected sample (e.g., plasma, feces, whole blood, saliva, milk, etc) in a buffer system containing anti-cortisol or anti-corticosterone antibody. The buffer system can also contain fluorescence suppressors or analyte specific assay modifiers;
b. Determine background of polarized fluorescence;
c. Add fluorescently-labeled peptide mimic. The concentration of the peptide mimic can be at a wide range, depending on assay conditions in order to ensure molecular interactive competition. However, in a preferred embodiment the range of peptide mimic concentration is 0.1 to 2 nM;
d. Measure the change in fluorescence polarization of the sample after the addition of labeled peptide mimic;
e. Calculate the background-subtracted polarization (anisotropy or millipolarization units) for each sample in a batch. For example, this can be accomplished by subtracting polarization before mimic addition (i.e., buffer system plus analytes and modifiers plus anti-cortisol antibody) from polarization of the same member from the same member of the batch from after addition of labeled-cortisol peptide mimic.
f. Calculate mP (e.g., vertical measurement after addition of mimic-vertical measurement before addition of mimic)—(horizontal measurement after addition of mimic-horizontal measurement before addition of mimic) plus horizontal measurement after addition of mimic plus horizontal measurement before addition of mimic;
g. Determine concentration of cortisol or corticosterone in sample.

### EXAMPLE 3

### Identification of Gamma-Interferon by Competitive FP Using Peptide Mimics

Although FP measurement of γ-interferon holds many advantages over other techniques, the use of FP is limited by the low availability of suitable competitors owing to the need for small molecular moieties. Isolated peptides can be used, which would markedly enhance FP assay operability.

However, γ-Interferon typically exists as a dimer composed of identical subunits. Therefore, anti-gamma-interferon antibody epitopes may encompass sites on both dimers, or even formed from the dimerization. To address the need of suitable competitive reagents for detection of γ-interferon by FP, we suitable polypeptides that could mimic the anti-γ-interferon epitopes were sought.

In order to identify peptide mimics, the procedure for described in Example 1 was followed. FA42™ anti-Gamma-interferon monoclonal antibody (Cellestis, Inc, Valencia, Calif.) was used in the screening of peptide mimics. Peptide-expressing phages that exhibited high affinity, as evidenced by ELISA, were further subjected to sequencing.

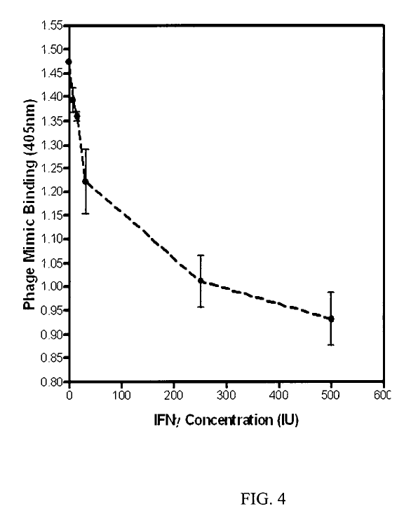

Identification of epitope sites that the peptides represented were then determined by comparing the peptide sequences with the known γ-interferon sequence. The identified peptides were then evaluated by the ability of gamma-interferon to inhibit binding of anti-γ-interferon antibody in ELISA. The results of this study is illustrated in FIG. 3 for peptide IFN-28. As shown in FIG. 4, a graded competition for binding to anti-gamma-interferon antibody using increasing amounts of gamma-interferon and a constant concentration of phage containing the peptide mimic. Phage binding was identified, in this study, using a specific anti-phage antibody.

Fluorescently tagged peptides were created based on the phage-displayed peptides isolated during the screening procedures using anti-γ-interferon antibody (FA42). Further analysis of the peptide mimics sought to ascertain whether these peptides operated in the same manner as they do when expressed on the surface of the phage. In order to ascertain if these peptides operated in the same manner as they do when expressed on the surface of the phage, a competition assay was performed. In this assay, the newly created peptides were used to compete with the peptides on the surface of the phage for binding to FA42.

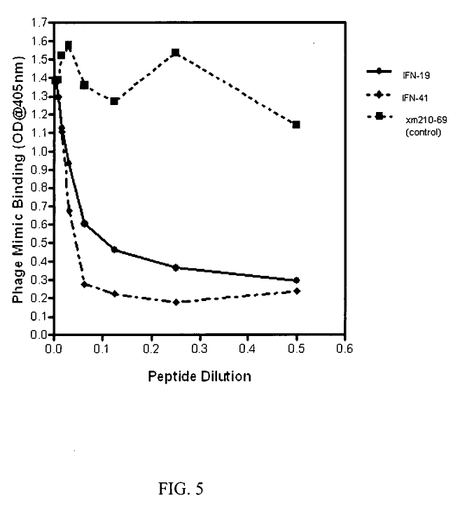

As illustrated in FIG. 4, both IFN-19 and IFN-41 competed with the IFN-41 expressing phage for binding to the anti-γ-interferon antibody. A gradient of inhibition of IFN-41 expressing phage binding by the two peptides is shown. The control peptide (ACA-20) is a mimic of cortisol. As expected, ACA-20 did not compete with the IFN-41 expressing phage for binding to the anti-γ-interferon antibody. Additionally, FIG. 5 illustrates gradient inhibition of IFN-41 and IFN-19 using anti-gamma-interferon antibody. In FIG. 5, the control peptide, ACA-20, is a mimic of cortisol and did not compete for antibody binding.

Table 3 lists the identified peptide sequences. The identified sequences can be generically described by the peptide motif T(S)−Q−Y(F/W)−x−Q(N)−Q(N). The motifs of the peptide mimics represented by SEQ ID No. 5, 6, 7, 8, 9, 10, 11, 12, 13, 15, 16 and 17 completely adhere to this basic motif.

These observations strongly suggest that these peptide mimics can be effective competing reagents in FP assays utilizing peptide mimics containing conformational epitopes of gamma interferon. The assay is capable of detecting not only gamma-interferon but also gamma-interferon dimers, if the appropriate antibody reagent is utilized.

TABLE 3

| | Peptide | |
|---|---|---|
| Mimic clone | Sequence | SEQ ID No. |
| IFN-67c | TAGTWYS | SEQ ID No. 1 |
| IFN-75 | GYPHWYH | SEQ ID No. 2 |
| IFN-4 | WQKRPPTPPLIS | SEQ ID No. 3 |
| IFN-61c | TPKQTTG | SEQ ID No. 4 |
| IFN-6c | SQYNRLV | SEQ ID No. 5 |
| IFN-9c | SQYNRLV | SEQ ID No. 6 |
| IFN-8c | SQYANSL | SEQ ID No. 7 |
| IFN-59c | SQWAHRL | SEQ ID No. 8 |
| IFN-37c | TQFNMQM | SEQ ID No. 9 |
| IFN-79c | TQFNIQM | SEQ ID No. 10 |
| IFN-41c | TQYEQHY | SEQ ID No. 11 |
| IFN-28c | SQYENNH | SEQ ID No. 12 |
| IFN-7c | SQYSHQI | SEQ ID No. 13 |
| IFN-19c | TQYSQQM | SEQ ID No. 14 |
| IFN-73c | SQYSQLY | SEQ ID No. 15 |
| IFN-55c | NQYNQQL | SEQ ID No. 16 |
| IFN-13c | SQYAQQQ | SEQ ID No. 17 |
| IFN-49 | IPQDRNH | SEQ ID No. 18 |
| IFN-57 | VKDRPSISYNLH | SEQ ID No. 19 |
| IFN-63c | TPGQHNL | SEQ ID No. 20 |
| IFN-40 | ATKMHIPLHSTR | SEQ ID No. 21 |
| IFN-65c | YNPNLKR | SEQ ID No. 22 |
| IFN-10c | QRAVFFQ | SEQ ID No. 23 |
| IFN-50 | KPVVSAHSAFA | SEQ ID No. 24 |

The general inventive FP method using peptide mimics, containing conformation epitopes of gamma-interferon, is as follows:
a. Incubate collected sample (e.g., plasma, feces, whole blood, saliva, milk, etc) in a buffer system containing anti-corticosteroid, such as cortisol or anti-corticosterone antibody. The buffer system can also contain fluorescence suppressors or analyte specific assay modifiers;
b. Determine background of polarized fluorescence;
c. Add fluorescently labeled peptide mimic. The concentration of the peptide mimic can be at a wide range, depending on assay conditions in order to ensure molecular interactive competition. However, in a preferred embodiment the range of peptide mimic concentration is 0.1 to 2 nM;
d. Measure the change in fluorescence polarization of the sample after the addition of labeled peptide mimic;
e. Calculate the background-subtracted polarization (anisotropy or millipolarization units) for each sample in a batch. For example, this can be accomplished by subtracting polarization before mimic addition (i.e., buffer system plus analytes and modifiers plus anti-cortisol antibody) from polarization of the same member from the same member of the batch from after addition of labeled-cortisol peptide mimic.
f. Calculate mP (e.g., vertical measurement after addition of mimic-vertical measurement before addition of mimic)—(horizontal measurement after addition of mimic-horizontal measurement before addition of mimic) plus horizontal measurement after addition of mimic plus horizontal measurement before addition of mimic;
g. Determine concentration of cortisol or corticosterone in sample.

### REFERENCES

1. Buchli, R., R. S. VanGundy, H. K. Hickman-Miller, C. F. Giberson, W. Bardet, and W. H. Hildebrand. Development and validation of a fluorescence polarization-based competitive peptide-binding assay for HLA-A 0201-A new tool for epitope discovery. Biochemistry 2005; 44:12491-12507.
2. Gallo D, George J R, Fitchen J H, Goldstein A S, Hindahl M S: Evaluation of a system using oral mucosal transudate for HIV-1 antibody screening and confirmatory testing. OraSure HIV Clinical Trials Group, JAMA 1997; 277:254-8.
3. Geysen, H. M., S. J. Rodda, T. J. Mason. A priori delineation of a peptide which mimics a discontinuous antigenic determinant. Mol. Immunol. 1986; 23:709-715.
4. Hirschman J D, Kresge J A: Synthesis of a symposium: innovative non- or minimally-invasive technologies for monitoring health and nutritional status in mothers and young children, J Nutr 2001; 131:1643 S-5S.
5. Hofman L F: Human saliva as a diagnostic specimen, J Nutr 2001; 131:1621 S-5S.
6. Kirschbaum, C. and D. H. Hellhammer: salivary cortisol in psychoneuroendocrine research: Recent developments and applications. Psychoneuroendocrinology, 1994; 19:313-333.
7. Kraus F, Konno J: The salivary secretion of antibody, Alabama J Med Sci 1965; 2:15-22.
8. Kowski T, Wu J: Fluorescence polarization is a useful technology for reagent reduction in assay miniaturization, Comb Chem High Throughput Screen 2000; 3:437-44.
9. O'Connor, K. H., C. Konigs, M. J. Rowley, J. A. Irving, L. C. Wijeyewickrema, A. Pustowka, U. Dietrich, I. R. Mackay. 2005. Requirement of multiple phage displayed peptide libraries for optimal mapping of a conformational antibody epitope on CCR5. J. Immunol. Methods 299:21-35.
10. Rossomando, E. F., Kousvelari, E., Janicki, B. W., Tabak, L. A. 2001. Improvement of oral health in the postgenomic era: opportunities for government/industry partnerships, Compend. Contin. Educ. Dent. 22: 570-4.
11. Rowley, M., O'Connor, K., Wijeyewickrema, L., 2004. Phage display for epitope determination: a paradigm for identifying receptor-ligand interactions. Biotechnol. Annu. Rev. 10, 151.
12. Schade, S., M. Jolley: U.S. Pat. No. 5,804,395 Fluorescence polarization assays of enzymes and substrates thereof, issued Sep. 8, 1998.
13. Tabak, L. A. 2001. A revolution in biomedical assessment: the development of salivary diagnostics. J. Dent Educ. 65: 1335-9.
14. Tencza S, Islam K, Kalia V, Nasir M, Jolley M, Montelaro R: Development of a fluorescence polarization-based diagnostic assay for equine infectious anemia virus, J Clin Microbiol 2000; 38:1854-9.
15. Terpetschnig E, Szmacinski H, Lakowicz J: Fluorescence polarization immunoassay of a high-molecular-weight antigen based on a long-lifetime Ru-ligand complex, Anal Biochem 1995; 227:140-7.
16. Thieme T, Piacentini S, Davidson S, Steingart K: Determination of measles, mumps, and rubella immunization status using oral fluid samples, JAMA 1994; 272:219-21.

Having described the invention, one of skill in the art will appreciate in the appended claims that many modifications and variations of the present invention are possible in light of the above teachings. It is therefore, to be understood that, within the scope of the appended claims, the invention may be practiced otherwise than as specifically described.

What is claimed is:

1. A competitive fluorescence polarization method comprising:
a. intermixing a sample suspected of containing a target antigen with a specific antibody to said compound and a competitive peptide mimic containing conformational epitopes of said target antigen;
b. indirectly measuring the amount of said target antigen by fluorescence polarization.

2. The competitive method of claim 1, wherein said target antigen is selected from the group consisting of a steroid, toxin, hormone, nucleic acid, protein and infectious organism.

3. The competitive method of claim 1, wherein said competitive peptide mimic is labeled.

4. The method of claim 1, wherein said sample is selected from the group consisting of food, environmental samples, and bodily fluids selected from the group consisting of saliva, oral rinse expectorant, oral fluid including oral mucosal transudate and gingival crevicular fluid, urine, sweat, tears, blood, serum, stool, gastric fluid, synovial fluid, phlegm, and other clinical and laboratory specimens and samples.

5. The competitive method of claim 2, wherein said infectious agent is a bacteria, virus, fungi or protozoa.

6. The competitive method of claim 2, wherein said steroid is a corticosteroid.

7. The competitive method of claim 2, wherein said protein is gamma-interferon or a dimer of gamma-interferon.

8. The competitive method of claim 6, wherein said corticosteroid is cortisol.

9. The competitive method of claim 6, wherein said cortisol peptide mimic has the sequence motif P+T+F(Y), W(F)+N(O) or L(I)+R(H/K).

10. The competitive method of claim 7, wherein said gamma-interferon peptide mimic is selected from the group consisting of SEQ ID No. 1-24.

11. The method of claim 3, wherein said label is a fluorochrome.

12. The competitive method of claim 7, wherein said gamma-interferon or said dimer of gamma-interferon peptide mimic has the sequence motif of hydroxylic-amidic-aromatic-x-amidic-amidic-(aliphatic or sulfur containing).

13. The competitive method of claim 8, wherein said cortisol peptide mimic is selected from SEQ ID No. 25-60.

14. The competitive method of claim 9, wherein said cortisol peptide mimic is selected from the group consisting of SEQ ID No. 25-35, SEQ ID No. 37-50, and SEQ ID No. 54-60.

15. The method of claim 11, wherein said fluorochrome is pH independent.

16. The method of claim 11 wherein said fluorochrome is selected from the group consisting of 7-AAD, Acridine Orange, Alexa 488, Alexa 532, Alexa 546, Alexa 568, Alexa 594, Aminonapthalene, Benzoxadiazole, BODIPY 493/504, BODIPY 505/515, BODIPY 576/589, BODIPY FL, BODIPY TMR, BODIPY TR, Carboxytetramethylrhodamine, Cascade Blue, a Coumarin, Cy2, CY3, CY5, CY9, Dansyl Chloride, DAPI, Eosin, Erythrosin, Ethidium Homodimer II, Ethidium Bromide, Fluorescamine, Fluorescein, FTC, GFP (yellow shifted mutants T203Y, T203F, S65G/S72A), Hoechst 33242, Hoechst 33258, IAEDANS, an Indopyras Dye, La Jolla Blue 3, a Lanthamide Chelate, a Lanthamide Cryptate, Lissamine Rhodamine, Lucifer Yellow, Maleimide, MANT, MQAE, NBD, Oregon Green 488, Oregon Green 514, Oregon Green 500, Phycoerythrin, a Porphyrin, Propidium Iodide, Pyrene, Pyrene Butyrate, Pyrene Maleimide, Pyridyloxazole, Rhodamine 123, Rhodamine 6G, Rhodamine Green, SPQ, Texas Red, TMRM, TOTO-1, TRITC, YOYO-1, vitamin B12, flavin-adenine dinucleotide, and nicotinamide-adenine dinucleotide.

17. The method of claim 11, wherein said fluorochrome concentration is 1 nM or less and the sample millipolarization is increased or decreased by at least 10 mP.

18. The competitive method of claim 12, wherein said gamma-interferon or gamma-interferon dimer peptide mimic is selected from the group consisting of SEQ ID No. 5; SEQ ID No. 6; SEQ ID No. 7; SEQ ID No. 8; SEQ ID No. 9; SEQ ID No. 10; SEQ ID No. 11; SEQ ID No. 12; SEQ ID No. 13; SEQ ID No. 15; SEQ ID No. 16; and SEQ ID No. 17.


SEQUENCE LISTING (60 sequences)

SEQ ID NO: 1 - protein, 8 aa, Homo sapiens
CTATWYSC

SEQ ID NO: 2 - protein, 7 aa, Homo sapiens
GTPHWYH

SEQ ID NO: 3 - protein, 12 aa, Homo sapiens
WQKRPPTPPLIS

SEQ ID NO: 4 - protein, 9 aa, Homo sapiens
CTPKQTTGC

SEQ ID NO: 5 - protein, 9 aa, Homo sapiens
CSQYNRLVC

SEQ ID NO: 6 - protein, 9 aa, Homo sapiens
CSQYNRLVC

SEQ ID NO: 7 - protein, 9 aa, Homo sapiens
CSQYANSLC

SEQ ID NO: 8 - protein, 9 aa, Homo sapiens
CSQWAHRLC

SEQ ID NO: 9 - protein, 9 aa, Homo sapiens
CTQFNMQMC

SEQ ID NO: 10 - protein, 9 aa, Homo sapiens
CTQFNIQMC

SEQ ID NO: 11 - protein, 9 aa, Homo sapiens
CTQYEQHYC

SEQ ID NO: 12 - protein, 9 aa, Homo sapiens
CSQYENNHC

SEQ ID NO: 13 - protein, 9 aa, Homo sapiens
CSQYSHQIC

SEQ ID NO: 14 - protein, 9 aa, Homo sapiens
CSQYSQLYC

SEQ ID NO: 15 - protein, 9 aa, Homo sapiens
CTQYSQQMC

SEQ ID NO: 16 - protein, 9 aa, Homo sapiens
CNQYNQQLC

SEQ ID NO: 17 - protein, 9 aa, Homo sapiens
CSQYAQQQC

SEQ ID NO: 18 - protein, 7 aa, Homo sapiens
IPQDRNH

SEQ ID NO: 19 - protein, 12 aa, Homo sapiens
VKDRPSISYNLH

SEQ ID NO: 20 - protein, 9 aa, Homo sapiens
CTPGQHNLC

SEQ ID NO: 21 - protein, 12 aa, Homo sapiens
ATKMHIPLHSTR

SEQ ID NO: 22 - protein, 9 aa, Homo sapiens
CYNPNLKRC

SEQ ID NO: 23 - protein, 9 aa, Homo sapiens
CQRAVFFQC

SEQ ID NO: 24 - protein, 11 aa, Homo sapiens
KPVVSAHSAFA

SEQ ID NO: 25 - protein, 11 aa, homo sapiens
ACMLSSQTLHC

SEQ ID NO: 26 - protein, 12 aa, homo sapiens
LFQTTVRTHALF

SEQ ID NO: 27 - protein, 7 aa, Homo sapiens
GYQPLRH

SEQ ID NO: 28 - protein, 12 aa, Homo sapiens
AHNLNYKPLRHP

SEQ ID NO: 29 - protein, 7 aa, Homo sapiens
KLMVPLH

SEQ ID NO: 30 - protein, 7 aa, Homo sapiens
FAHPLYP

SEQ ID NO: 31 - protein, 12 aa, Homo sapiens
FVPSPHRPLYQP

SEQ ID NO: 32 - protein, 10 aa, Homo sapiens
ACTQFNLHYC

SEQ ID NO: 33 - protein, 12 aa, Homo sapiens
WPFTYHMPTHYG

SEQ ID NO: 34 - protein, 12 aa, Homo sapiens
LPYNVHWAKGRP

SEQ ID NO: 35 - protein, 12 aa, Homo sapiens
GPSTFFSSGLLR

SEQ ID NO: 36 - protein, 7 aa, Homo sapiens
MGPSNMY

SEQ ID NO: 37 - protein, 10 aa, Homo sapiens
ACPATSIRMC

SEQ ID NO: 38 - protein, 7 aa, Homo sapiens
SQNIFRW

SEQ ID NO: 39 - protein, 12 aa, Homo sapiens
QNNHMSSRQWFA

SEQ ID NO: 40 - protein, 10 aa, Homo sapiens
ACSQFNSLFC

SEQ ID NO: 41 - protein, 10 aa, Homo sapiens
ACSQYNRLVC

SEQ ID NO: 42 - protein, 10 aa, Homo sapiens
ACTLPWSNSC

SEQ ID NO: 43 - protein, 12 aa, Homo sapiens
TNLYQDKPSNRS

SEQ ID NO: 44 - protein, 12 aa, Homo sapiens
LWPRSDSHIPAF

SEQ ID NO: 45 - protein, 12 aa, Homo sapiens
KNWNTPTYYSSM

SEQ ID NO: 46 - protein, 12 aa, Homo sapiens
SKNWHTPTYSTL

SEQ ID NO: 47 - protein, 7 aa, Homo sapiens
LSKNWQT

SEQ ID NO: 48 - protein, 10 aa, Homo sapiens
ACSQWSNVLC

SEQ ID NO: 49 - protein, 10 aa, Homo sapiens
ACTQWSQLYC

SEQ ID NO: 50 - protein, 12 aa, Homo sapiens
WYSPEYYVLPSL

SEQ ID NO: 51 - protein, 11 aa, Homo sapiens
ACPPPWHTMKC

SEQ ID NO: 52 - protein, 10 aa, Homo sapiens
ACVNPNLKVC

SEQ ID NO: 53 - protein, 10 aa, Homo sapiens
ACINPNDKKC

SEQ ID NO: 54 - protein, 12 aa, Homo sapiens
TASLTKTSVNPK

SEQ ID NO: 55 - protein, 12 aa, Homo sapiens
YFTPVFINPTQV

SEQ ID NO: 56 - protein, 12 aa, Homo sapiens
LPKSSLQALQLR

SEQ ID NO: 57 - protein, 12 aa, Homo sapiens
LQVNLEQDKLRP

SEQ ID NO: 58 - protein, 12 aa, Homo sapiens
LDLRQNSSPPLR

SEQ ID NO: 59 - protein, 11 aa, Homo sapiens
LHTQHAFTEGN

SEQ ID NO: 60 - protein, 11 aa, Homo sapiens
HVNSSISGQLL